(19) United States
(12) Patent Application Publication
(10) Pub. No.: US 2018/0209971 A1
(43) Pub. Date: Jul. 26, 2018

(54) TEST STRIP FOR DIAGNOSTIC TESTING

(71) Applicant: ApolloDX, LLC, Salt Lake CIty, UT (US)

(72) Inventors: William Robert Pagels, Salt Lake City, UT (US); Jared B. Bauer, Salt Lake City, UT (US)

(21) Appl. No.: 15/747,116

(22) PCT Filed: Jul. 28, 2016
(86) PCT No.: PCT/US2016/044424

§ 371 Date: Jan. 23, 2018

Related U.S. Application Data
(60) Provisional application No. 62/198,041, filed on Jul. 28, 2015.

(51) Int. Cl. G01N 33/543; B01L 3/00

(52) U.S. Cl. CPC G01N 33/54366; B01L 2300/0861; B01L 2300/0816; B01L 3/502746

(57) ABSTRACT

A test strip includes a body defining a sample entry, a test area, and a cavity. The cavity is configured to transport a test sample from the sample entry to the test area. The test strip includes at least one passive mixing element within the cavity. The passive mixing element is configured to cause passive mixing of the test sample as the test sample is carried from the sample entry toward the test area. A test sample to be analyzed is introduced to the sample entry and at least a portion of the test sample is transported through the cavity to the test area. During transport, the test sample is passively mixed.

### CROSS-REFERENCE TO RELATED APPLICATIONS

This application is a national phase entry under 35 U.S.C. § 371 of International Patent Application PCT/US2016/044424, filed Jul. 28, 2016, designating the United States of America and published in English as International Patent Publication WO 2017/019844 A1 on Feb. 2, 2017, which claims the benefit under Article 8 of the Patent Cooperation Treaty of the filing date of U.S. Provisional Patent Application Ser. No. 62/198,041, filed Jul. 28, 2015, for “Test Strip for Diagnostic Testing,” the entire disclosure of which is hereby incorporated by reference.

### TECHNICAL FIELD

Embodiments of the application relate generally to test devices for diagnostic samples, such as biological samples, and associated methods of making and using such devices.

### BACKGROUND

Glucometers typically use test strips having similar external interfaces and internal topography. However, due to the high concentration of glucose in blood samples, no particular exogenous constituents of the assay milieu are needed in order to produce diagnostic test results of sufficient sensitivity for clinical monitoring purposes. Glucose test strips are usually multi-laminar devices in which a middle layer of the multi-laminar format is cut so that when sandwiched between layers above and below, a hollow chamber is formed that can be filled by capillary action, wicking blood from the sample point of entry at one end of the test strip to the site where an electrochemical measurement is performed at the end of the strip distal from the sample entry point.

For example, test strips are described in U.S. Pat. No. 5,951,863, “Disposable Glucose Test Strip and Method and Compositions for Making Same,” issued Sep. 14, 1999; U.S. Pat. No. 5,426,032, “No-wipe Whole Blood Glucose Test Strip,” issued Jun. 20, 1995; U.S. Patent Application Publication 2004/0087034, “Test Strip,” published May 6, 2004; and U.S. Patent Application Publication 2013/0266481, “Blood Glucose Test Strip,” published Oct. 10, 2013. The entire contents of each of these documents are hereby incorporated by reference.

### BRIEF SUMMARY

Described herein is a test device (e.g., a consumable device) that can be used to mix a diagnostic sample (including, but not limited to, blood, urine, saliva, or solid samples dissolved in an aqueous medium, etc.) with constituents of an assay milieu suitable for producing a diagnostic test result. This test device includes a chamber that is filled either by passive capillary action or exerted through an active external force outside of the test device. In either case, upon entry in the test device, the sample soon comes in contact with soluble salts or other assay components to produce a detectable signal. Upon further migration in the test device, the sample and solubilized salts become mixed as a consequence of moving over, under and/or around fluid path impediments of various configurations, locations, and sizes. Because of the intervention of these passive mixing elements, by the point of the fluid's migration to the “read zone” or “detection zone” where the sample is interrogated by the diagnostic system, the sample and assay components are suitably mixed to produce the desired test result.

In some embodiments, a test strip includes a body defining a sample entry, a test area, and a cavity. The cavity is configured to transport a test sample from the sample entry to the test area. The test strip includes at least one passive mixing element within the cavity. The at least one passive mixing element is configured to cause passive mixing of the test sample as the test sample is carried from the sample entry toward the test area.

A method of utilizing a test strip includes introducing a test sample to be analyzed to a sample entry at a first end of a test strip and transporting at least a portion of the test sample through a cavity from the sample entry to a test area. The test strip defines the cavity between the sample entry and the test area, which is at a second, distal end of the test strip. Because the test strip includes or contains at least one passive mixing element within the cavity, the test sample is passively mixed during transport through the test strip.

A method of forming a test strip includes forming a body defining a sample entry, a test area, and a cavity, wherein the cavity is configured to transport a test sample from the sample entry to the test area; providing at least one passive mixing element within the cavity; and configuring the at least one passive mixing element to cause passive mixing of the test sample as the test sample is carried from the sample entry toward the test area through the cavity.

### BRIEF DESCRIPTION OF THE DRAWINGS

FIG. 1A is a simplified cross-sectional top view of a middle layer of a test strip in one configuration.

FIG. 1B is a simplified perspective exploded view of the test strip of FIG. 1A.

FIG. 1C is a simplified cross-sectional side view of the test strip shown in FIGS. 1A and 1B.

FIGS. 2-11A are simplified cross-sectional top views of other embodiments of middle layers of test strips.

FIG. 11B is a simplified cross-sectional side view of the test strip shown in FIG. 11A.

FIGS. 12-15 are simplified cross-sectional side views of embodiments of test strips.

### DETAILED DESCRIPTION

The illustrations presented herein are not actual views of any particular test strip or sample detector, but are merely idealized representations that are employed to describe example embodiments of the present disclosure. Additionally, elements common between figures may retain the same numerical designation.

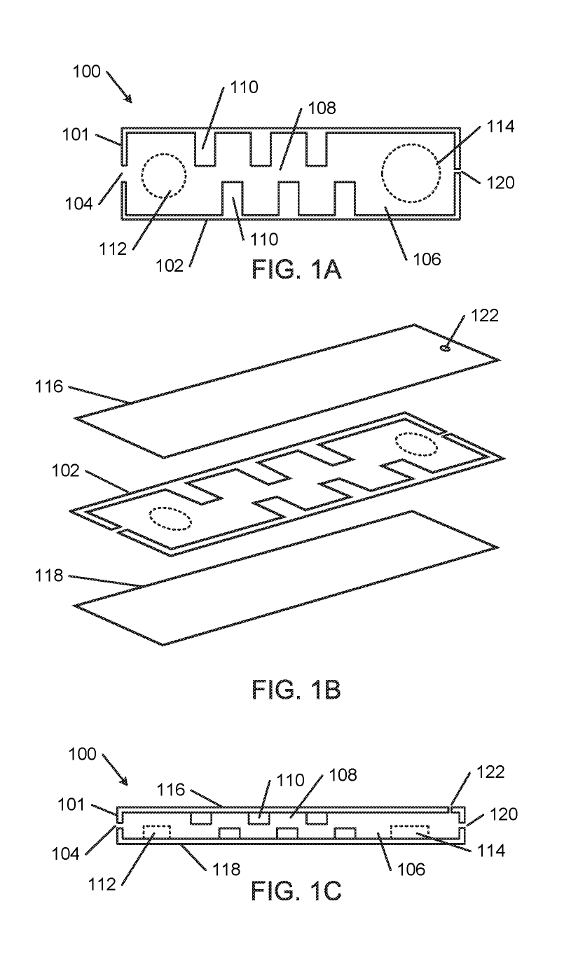

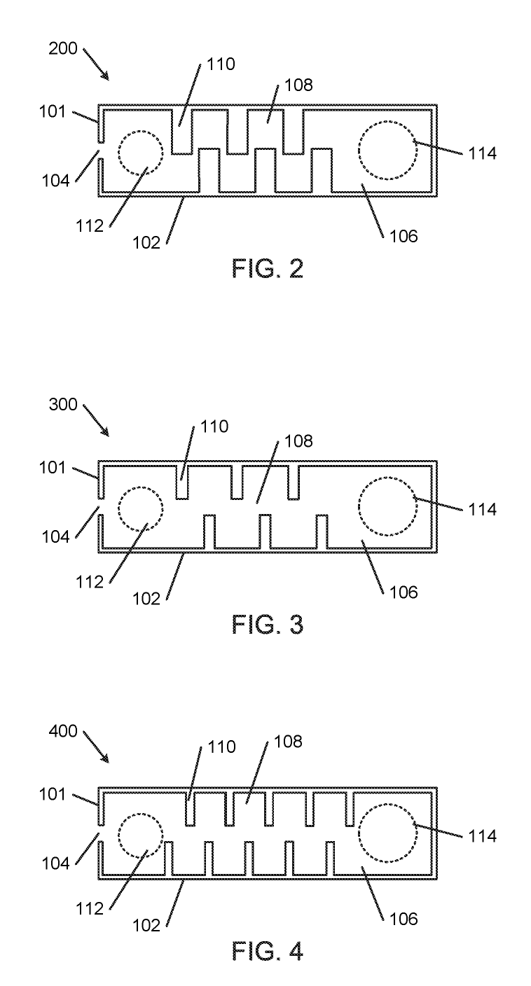

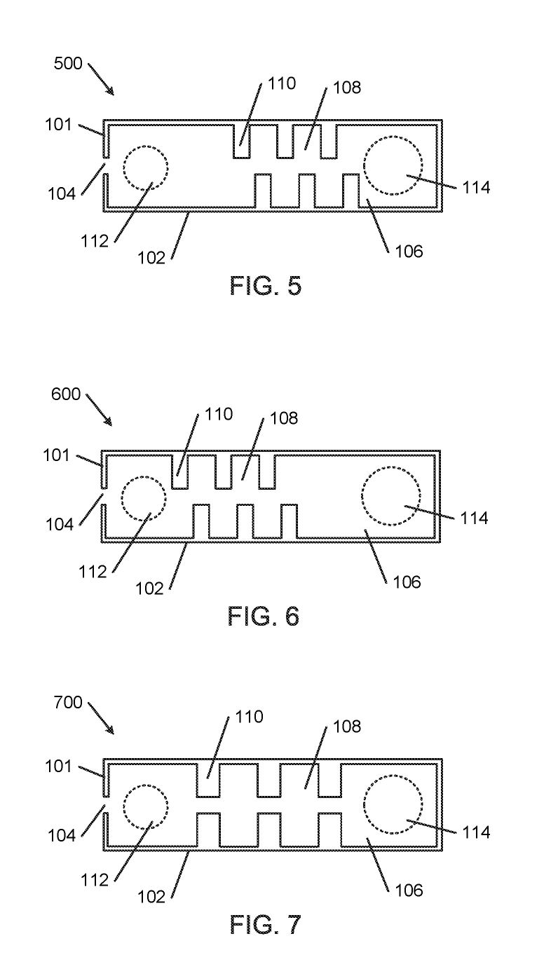

FIGS. 1A-1C show an embodiment of a test strip 100, which may be used for analyzing chemical or biological analytes. For example, the test strip 100 may be used for mixing a sample as the sample travels within the test strip 100.

As shown in FIG. 1A, the test strip 100 includes a body 101 configured to contain a test sample. The body 101 may include an outer wall 102, and may define a sample entry 104 (e.g., as an opening within the outer wall 102. The body 101 may also include an upper wall 116 (e.g., a top layer or roof), a lower wall 118 (e.g., a bottom layer or floor), or both, as shown in exploded view in FIG. 1B. In some embodiments, the outer wall 102 may be integral with the upper wall 116, the lower wall 118, or both. The upper wall 116 and/or the lower wall 118 may be layers or plates surrounding the outer wall 102. In other embodiments, the upper wall 116, the lower wall 118, or both may be bonded or laminated to the outer wall 102. The body 101 may be formed by injection molding, additive manufacturing (commonly referred to as “3D printing”), or any other selected process.

The body 101 may further define a test area 106 and a mixing cavity 108. As a test sample travels from the sample entry 104 through the mixing cavity 108 to the test area 106, the sample may be mixed to give the test sample a more uniform composition at the test area 106 than at the sample entry 104.

The sample entry 104 may be configured to receive a test sample via capillary action. In other embodiments, the sample entry 104 may be configured to receive a test sample via pressure exerted by an external force. For example, a force external to the test strip 100 may drive the test sample from the sample entry 104 to the test area 106.

In some embodiments, the sample entry 104 may have a maximum linear dimension (e.g., a diameter, a width, etc.) from about 0.1 mm to about 5.0 mm, such as from about 0.5 mm to about 3.0 mm or from about 1.0 mm to about 2.0 mm. The size of the sample entry 104 may be selected based at least in part on the method of receiving the test sample (e.g., by capillary action or a driving external force), the expected composition of the test sample, whether the test sample is expected to contain solids, or other factors.

In some embodiments, the outer wall 102 may include an orifice 120, shown in FIG. 1A, to provide a path for air to escape from the mixing cavity 108 and relieve back pressure from the capillarity-dependent filling of the mixing cavity 108. In some embodiments, the upper wall 116, or the lower wall 118 may include an orifice 122 instead of or in addition to the orifice 120. The orifice 120, 122 may be configured to prevent the escape of test sample. For example, the orifice 120, 122 may have a width or diameter of less than about 1.0 mm, less than about 0.1 mm, or even less than about 0.01 mm. In some embodiments, the orifice 120, 122 may be sealed prior to mixing.

The mixing cavity 108 in the test strip 100 is configured to transport the test sample from the sample entry 104 to the test area 106 while mixing the test sample. As shown in FIG. 1A, the test strip 100 may include one or more passive mixing elements 110 within or partially defining the mixing cavity 108. The passive mixing elements 110 may be configured as partial barriers within the test strip 100, such that the test sample must travel around, over, under, and/or through the passive mixing elements 110 to reach the test area 106.

For example, the test sample may be mixed by turbulence caused by interruption of flow of the test sample by the passive mixing elements 110 within the mixing cavity 108. The test strip 100 may include rows of passive mixing elements 110. For example, the test strip 100 may include a first row of passive mixing elements 110 connected to a first lateral surface of the mixing cavity 108 (depicted as the row of three shown at the top of FIG. 1A) and a second row of passive mixing elements 110 connected to a second, opposite lateral surface of the mixing cavity 108 (depicted as the row of three shown at the bottom of FIG. 1A). In some embodiments, the test strip 100 may include a first row of passive mixing elements 110 connected to a top surface of the mixing cavity 108 (depicted as the row of three shown at the top of FIG. 1C) and a second row of passive mixing elements 110 connected to a bottom surface of the mixing cavity 108 (depicted as the row of three shown at the bottom of FIG. 1C). The passive mixing elements 110 may be shorter than the mixing cavity 108, such that the test sample can travel over and around the passive mixing elements 110. In some embodiments, the passive mixing elements 110 may be as tall as the mixing cavity 108, such that the test sample must travel around the passive mixing elements 110 to reach the test area 106.

The passive mixing elements 110 may be offset from one another or in a staggered pattern to cause flow around the passive mixing elements 110 and through the spaces in between. The passive mixing elements 110 may each have substantially the same dimensions, or may have different dimensions from one another. The passive mixing elements 110 may be integral with the outer wall 102 of the test strip 100.

The test strip 100 may include a reagent 112 useful for a diagnostic reaction (a “diagnostic reagent”) adjacent the sample entry 104 and configured to mix with the test sample as the test sample travels through the test strip 100. For example, the reagent 112 may be in the form of a dissolvable solid, such as a bead. The reagent 112 may be readily soluble in test samples expected to be used with the test strip 100. The reagent 112 may be a non-physiological chemical compound such as a dye, a derivatized dye, or a synthesized molecule formulated to block binding pairs of molecules that may otherwise interfere with a binding event that produces a diagnostic signal. In some embodiments, the reagent 112 may include an inorganic salt, a buffer salt (which may be organic or inorganic), a binder, an interferant, a proteinaceous component, a catalytic component, a nucleic acid, etc.

The passive mixing element(s) 110 may be configured to promote mixing of the reagent 112 with the test sample to produce test sample suitable for testing at the test area 106. The reagent 112 may react with one or more components of the test sample during mixing. For example, the reagent 112 may facilitate or amplify electron transfer between a distal electrochemical tethered to a polymer and an electrode to which the polymer is attached. In another example, the reagent 112 may be an enzymatic substrate used in the diagnostic reaction to produce a measureable signal related to the presence of the target in the test sample.

The reagent 112 is depicted as a discrete circle from the top view (FIG. 1A) and a rectangle in cross section (FIG. 1C). The reagent 112 may be of any configuration or may occupy the entire or some portion of the space on either the top layer 116 or bottom layer 118 of the test strip 100 in any of the variations of the test strip disclosed herein.

In certain embodiments, the test strip 100 may include a sensor 114 configured to detect a composition of at least one analyte in the mixture without further mixing. That is, the test strip 100, including the passive mixing elements 110, may mix the test sample sufficiently for the sensor 114 to provide a measure of the amount of the analyte in the test sample.

The sensor 114 may be or include an electrochemical sensor, a fluorescent sensor, a chemiluminescent detector, a colorimetric sensor, etc. As an example of one of these detection systems, the sensor 114 may include or be an electrode as a part of a system by which presence of a chemical entity creates an increase or decrease in an electrical signal, which may be accomplished by the presence of chemical capture probes bound to the electrode. The capture probes may bind the reagent (e.g., a reagent comprising a redox conjugate), the chemical entity of interest, or a combination thereof, which may cause either an increase in the transfer of electrons to the electrode or sensor, a decrease in the transfer of electrons to the electrode or sensor, or a combination thereof. This electrochemical system may include, but is not limited to, the chemical entity of interest, the reagent (e.g., a reagent comprising redox conjugates), capture probes, or other compounds known to one of ordinary skill in the art.

The increase or decrease in the electrical signal may be transferred through this electrochemical system to the electrodes or sensors, which may pass the electrical signal to another device, either wirelessly or through one or more wires. The signal may be quantified through measurement by a voltmeter, an ammeter, or another electronic measurement device. Electrical sensors are described in, for example, U.S. Patent Application Publication 2015/0006089 A1, “Devices, Systems, and Methods for Diagnostic Testing,” published Jan. 1, 2015; and U.S. Patent Application Publication 2015/0268186 A1, “Systems and Methods for Diagnostic Testing,” published Sep. 24, 2015; the entire disclosures of each of which are hereby incorporated by reference.

In some embodiments, the electrode or other sensor may be bound and/or coupled to capture probes, which may include a peptide, an oligomer, and/or another chemical or biological entity. The chemical entity may allow indirect and/or direct binding of the peptide to the electrode. For example, the chemical entity may comprise a thiolated hydrocarbon chain, which may be bound to the N-terminus of a peptide. The C-terminus of the peptide may be modified and bound with a plurality of chemical agents including, but not limited to, a redox agent such as methylene blue. In some embodiments, the peptide may have a chemical affinity for one or multiple entities in the sample solution. When there is no bond between these entities and the peptide, the peptide may be highly flexible, and may efficiently achieve electron transfer to and from the redox agent. When there is a bond between these entities and the peptide, the peptide may lose the ability or efficiency of electron transfer to and from the redox agent through a plurality of mechanisms including, but not limited to, being physically and chemically obstructed by the bound entity, or moved a sufficient distance away from the electrode. In some embodiments, the test strip 100 also includes a solution that is capable of unbinding the peptide from the entity.

In other embodiments, the sensor 114 may include a DNA sensor such as, in some embodiments, an aptamer. In such embodiments, the electrical conductivity of DNA and/or other oligonucleotide constructs can be dependent on its conformational state, its proximity to neighboring tethered aptamers or the complementariness of nucleic acids in the DNA sequence. For example, upon binding or otherwise incorporating an analyte from a sample, the conformation of the DNA sensor may switch, thereby resulting in an altered conductive path between two oligonucleotide stems. An electrode or other sensor may be used to monitor the electron transfer. This methodology of electrochemical detection is further described in U.S. Pat. No. 7,947,443, “DNA and RNA Conformational Switches as Sensitive Electronic Sensors of Analytes,” issued May 24, 2011; and U.S. Pat. No. 7,943,301, “DNA Conformational Switches as Sensitive Electronic Sensors of Analytes,” issued May 17, 2011, the entire disclosure of each of which is hereby incorporated by reference.

In other embodiments, the detection method may comprise colorimetry, luminescence, electrochemiluminescence, fluorimetry or other quantitating, sensing methodology. For example, the test strip 100 may be configured to be used with a diagnostic device such as a colorimeter or a fluorimeter. The colorimeter or fluorimeter may be coupled to other components, and may be used to analyze various sample types.

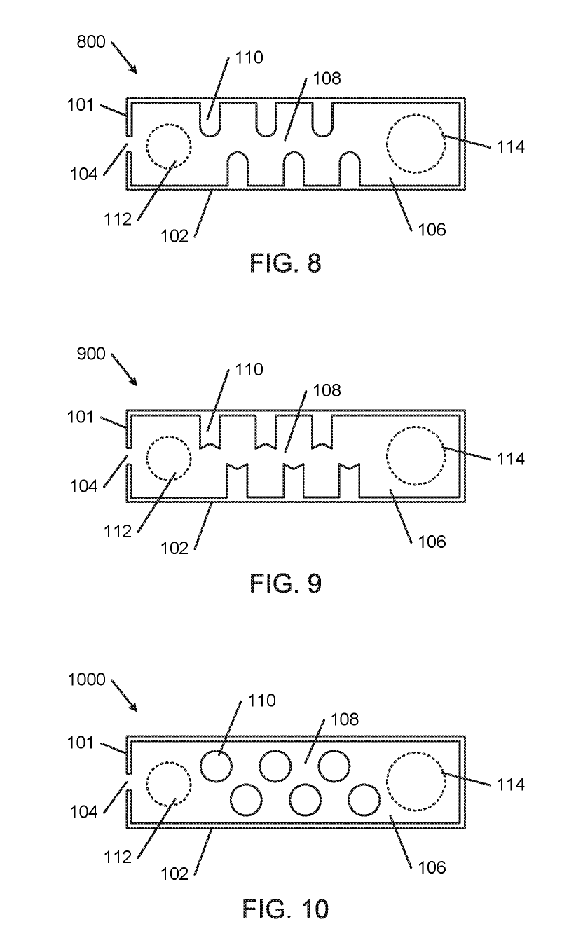

The passive mixing elements 110 may be arranged in any selected configuration. For example, FIG. 2 shows a test strip 200 having larger passive mixing elements 110 than the test strip 100 of FIG. 1A. The passive mixing elements 110 may extend into the space between the passive mixing elements 110 on the opposite side of the mixing cavity 108.

FIG. 3 shows a test strip 300 having narrower passive mixing elements 110 than the test strip 100 of FIG. 1A. Thus, the minimum spacing in the mixing cavity 108 between adjacent passive mixing elements 110 in the test strip 300 of FIG. 3 may be relatively larger than in the test strip 100 of FIGS. 1A-1C.

FIG. 4 shows a test strip 400 having narrower passive mixing elements 110 and more passive mixing elements 110 than the test strip 100 of FIG. 1A. Though shown as having ten passive mixing elements 110, the test strip 400 may have any number of passive mixing elements 110.

FIG. 5 shows a test strip 500 in which the passive mixing elements 110 are closer to the test area 106 than in the test strip 100 of FIG. 1A. Thus, the volume near the sample entry 104 and the reagent 112 (if present) may be relatively larger than in the test strip 100. In some embodiments, and as shown in FIG. 6, the passive mixing elements 110 may be located closer to the sample entry 104 of a test strip 600, leaving a larger, unobstructed space without passive mixing elements adjacent to the test area 106.

FIG. 7 shows a test strip 700 passive mixing elements 110 directly opposite one another. The test sample may tend to spread out after passing between the opposing pairs of passive mixing elements 110.

FIG. 8 shows a test strip 800 in which the passive mixing elements 110 have rounded surfaces. FIG. 9 shows a test strip 900 in which the passive mixing elements 110 have one or more obtuse external angles. FIG. 10 shows a test strip 1000 having rounded passive mixing elements 110 separated from the outer wall 102. In other embodiments, the passive mixing elements 110 may be elliptical, triangular, square, or any other selected shape. The shape and arrangement of the passive mixing elements 110 may affect the flow and mixing of the test sample. Therefore, different shapes and arrangements may be selected for different applications.

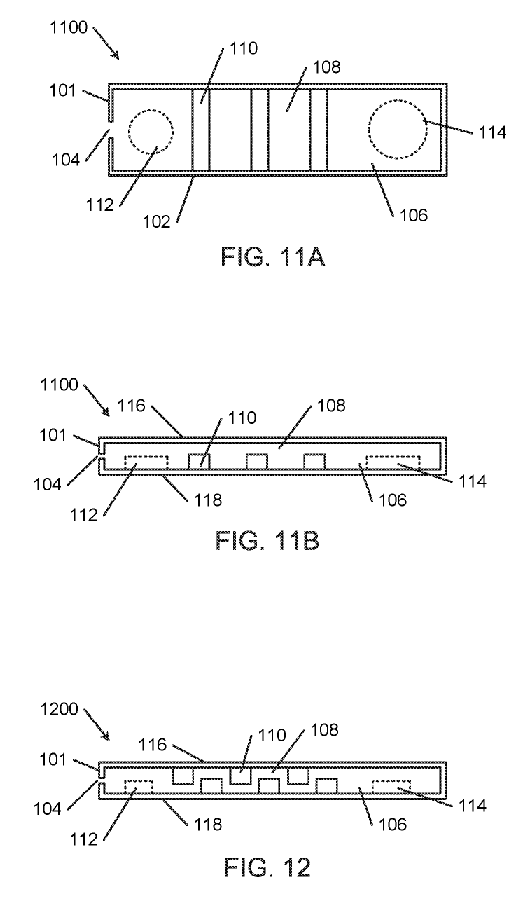

FIGS. 11A and 11B show a test strip 1100 having passive mixing elements 110 that span the width of the test strip 1000. FIG. 11B shows a cross-sectional side view of the test strip 1100 in which the passive mixing elements 110 are all connected to the lower wall 118, but some or all of the mixing elements 110 may be connected to the upper wall 116. To allow the test sample to pass, these passive mixing elements 110 may be shorter than the mixing cavity 108 itself. Thus, the test sample may flow over or under these passive mixing elements 110.

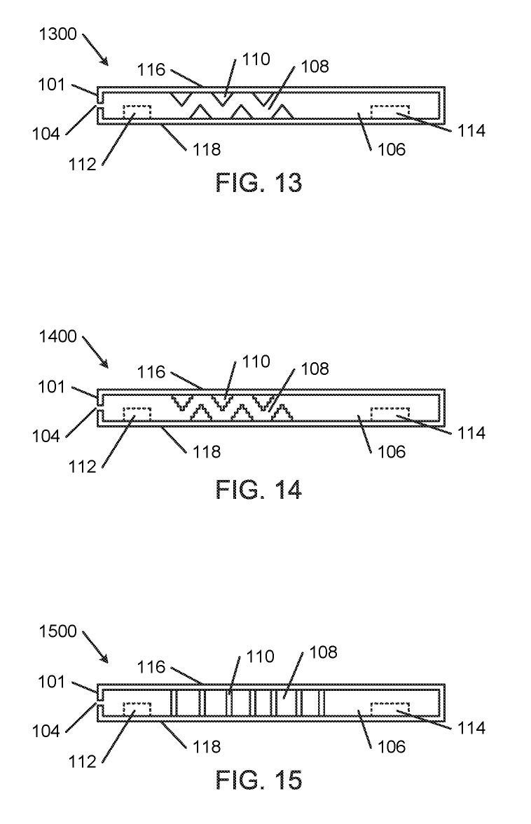

FIG. 12 shows a cross-sectional side view of a test strip 1200. The test strip 1200 has taller passive mixing elements 110 than the test strip 100 of FIG. 1C. FIG. 13 shows a cross-sectional side view of a test strip 1300 in which the passive mixing elements 110 have a triangular cross section. FIG. 14 shows a cross-sectional view of a test strip 1400 in which the passive mixing elements 100 have a cross section composed of steps, such as may be introduced by successive layering in a manufacturing process. FIG. 15 shows a cross-sectional side view of a test strip 1500 in which the passive mixing elements 110 span the entire height of the mixing cavity 108. In such embodiments, the passive mixing elements 110 would not span the entire width, so that the test sample can pass from the sample entry 104 to the test area 106.

The shapes of the passive mixing elements 110 shown in top view in FIGS. 2-10 may be combined with the shapes of the passive mixing elements 110 shown in side view in FIGS. 12-14 in any combination, so long as a fluid path remains from the sample entry 104 to the test area 106. The test strips 100-1400 shown in FIGS. 1-14 are merely examples of configurations that may be used. The number, size, shape, and placement of the passive mixing elements 110 affect how the test sample is mixed. Furthermore, the dimensions of the test strips themselves may vary based on the passive mixing elements 110 contained therein and the test sample to be analyzed.

The test strips 100-1400 shown in FIGS. 1-14 can be constructed by, for example, forming the outer wall 102 and bonding the upper wall 116 and the lower wall 118 to the outer wall 102. In some embodiments, the test strips may be formed of a single unitary sheet of material with the passive mixing elements 110 integrated with the outer wall 102, the upper wall 116, and/or the lower wall 118. In some embodiments, a layer of material with barrier elements may be injection molded, 3D printed, laminated, or prepared by some other process. In other embodiments, each layer of the test strip as depicted may include additional layers or portions with adhesive on the top, bottom, and/or side (e.g., between layers). For example, the outer wall 102 may include two or more parts joined together, such as a left side and a right side, joined together near a longitudinal axis.

In some embodiments, a method of utilizing the test strips 100-1400 shown in FIGS. 1-14 includes introducing a test sample to be analyzed to the sample entry 104 at a first end of the test strip 100. As discussed above, the test strip 100 defines a mixing cavity 108 between the sample entry 104 and the test area 106, which may be at a second, distal end of the test strip 100. The test sample introduced may be a liquid sample, such as a biological liquid (e.g., blood, urine, saliva, etc.) or an environmental liquid (e.g., water, brine, oil, etc.). In some embodiments, the test sample may be a solution or suspension used to extract material from a swab used to test for anthrax, environmental pollutants, or explosives on a surface. In some embodiments, the test solution or suspension may be prepared from an air collection sample including particulates, biological species, and/or gaseous molecules.

After the test sample is introduced through the sample entry 104, at least a portion of the test sample may be transported through the mixing cavity 108 and passively mixed while traveling through the mixing cavity 108. That is, the test sample may be mixed without motion of the mixing cavity 108 or the test strip 100 itself—the only motion may be the motion of fluids in the test strip 100. At least a portion of the test sample may flow around at least a portion of the passive mixing element(s) 110. The test sample may flow by capillary action, without application of an external mechanical force.

To form the test strips 100-1400 shown in FIGS. 1-14, the body 101 may be formed to define the sample entry 104, the test area 106, and the mixing cavity 108, and the mixing cavity 108 may be configured to transport a test sample from the sample entry 104 to the test area 106. The passive mixing elements 110 may be formed in or provided within the mixing cavity 108. The passive mixing elements 110 are configured to cause passive mixing of the test sample as the test sample is carried from the sample entry 104 toward the test area 106 through the mixing cavity 108. As discussed above, the body 101 may be formed by injection molding, additive manufacturing, or any other selected process.

In some embodiments, the reagent 112 is disposed adjacent the sample entry 104, such as by spraying, pipetting, or otherwise transferring the reagent 112 to the body 101. The reagent 112 may be further processed, such as by drying or lyophilizing.

The test strips 100-1400 may provide users the ability to accurately and precisely mix constituents of a specialized assay milieu with the test sample. Thus, the test strips 100-1400 may yield more repeatable, sensitive, specific, and/or stable test results for samples that require a reagent 112 than conventional testing methods. In contrast with, for example, blood glucose testing, in which the blood itself may be sufficient as the assay milieu, a preselected amount of the reagent 112 may help produce a solution in which various test samples behave in a known manner (e.g., a certain molecule or biological component may produce a corresponding diagnostic response).

Mixing of the test sample with the reagent 112 may result in assay conditions for diagnostic detection having greater sensitivity (by orders of magnitude, in some instances) than the sensitivity typically required for glucose detection. The test sample may include a biological material, such as blood, urine, or saliva, or an environmental sample on, for example, a test swab. The test sample may include a solid material mixed with a liquid (e.g., a suspension), or may be entirely liquid (e.g., a solution). To use the test strips 100-1400 herein to test a suspension, solid material (e.g., solid particles) may be first mixed with a liquid (e.g., a buffer, water, an alcohol, etc.).

The motive force translating the test sample through the test strips 100-1400 may be either passive (e.g., through capillary action) or active (i.e., through some external force imparted to the sample). In either case, the test sample may contact the reagent 112 (chemical, typically non-physiological and/or biological components) a short distance from the sample entry 104 to form an assay mixture. The reagent 112 can include buffering salts, inorganic salts that can catalyze or facilitate signal generation for detection systems, inhibitors of undesired reactions, or binders to interferents in a desired chemical detection scheme. The reagent 112 may also contain control analytes to help a detection system determine that either adequate sample was provided to the test area 106 or to assure that the system is working as intended. Thus, the test strips 100-1400 may include built-in means to validate the test result of an unknown in the test sample. These components can be used to produce measurable signals for detection including, but not limited to, colorimetric, electrochemiluminescent, chemiluminescent, luminescent, fluorescent, or electrochemical analyses.

In use, the test sample and the non-physiological assay components' progress toward the test area 106 is impeded by the passive mixing elements 110, which provide physical barriers. A purpose of the passive mixing elements 110, regardless of their conformation or configuration, is to promote hydrodynamic turbulence sufficient to retard some portions of the sample-assay component solution while not retarding other portions. The result is typically mixing of this solution. Turbulence occurs in the form of back eddies, retardation of fluid near solid surfaces, bending of fluid around obstacles, topographical shape, spacing and dimensions of the obstacles, and proximity of these elements to the test area 106. While the entire fluid column—from the sample entry 104 to the test area 106—will not typically be homogeneous with respect to the mixing of the reagent 112 with the sample, the concentrations of assay components at the test area 106 may nonetheless be consistent from one test strip 100-1400 to other similarly configured test strips.

The reagent 112 near the sample entry 104 may be in various forms. The reagent 112 can be a material in the form of a lyophilized bead, a deposited then evaporated material, or a lyophilized layer of assay components on the surface of the body 101. The reagent 112 may include more than one component. Once dissolved in the sample, assay components may be mixed before reaching the test area 106.

1. A test strip, comprising:
a body defining a sample entry, a test area, and a cavity, wherein the cavity is configured to transport a test sample from the sample entry to the test area; and
a dissolvable bead containing at least one reagent adjacent the sample entry; and
at least one passive mixing element within the cavity, the at least one passive mixing element configured to cause passive mixing of the test sample with the at least one reagent as the test sample is carried from the sample entry toward the test area.

2. (canceled)

3. The test strip of claim 1, further comprising a sensor configured to detect a composition of at least one analyte in the mixture without further mixing.

4. (canceled)

5. The test strip of claim 1, wherein the at least one reagent is readily soluble in the test sample.

6. The test strip of claim 1 wherein the at least one reagent comprises a non-physiological chemical compound.

7. The test strip of claim 1, wherein the at least one reagent comprises at least one material selected from the group consisting of buffer salts, inorganic salts, proteinaceous components, catalytic components, and nucleic acids.

8.-10. (canceled)

11. The test strip of claim 1, wherein the at least one passive mixing element is configured such that the test sample is mixed by turbulence caused by interruption of the flow of the test sample from the sample entry to the test area.

12. The test strip of claim 1, wherein the at least one passive mixing element comprises at least two rows of solid barriers.

13. The test strip of claim 12, wherein the at least two rows of solid barriers are arranged in a staggered pattern.

14. The test strip of claim 12, wherein at least one row of solid barriers is connected to a top surface of the cavity, and wherein at least another row of solid barriers is connected to a bottom surface of the cavity.

15. The test strip of claim 12, wherein at least one row of solid barriers is connected to a first lateral surface of the cavity, and wherein at least another row of solid barriers is connected to a second, opposite lateral surface of the cavity.

16. (canceled)

17. The test strip of claim 1, wherein the at least one passive mixing element comprises at least one barrier defining an obtuse external angle.

18. The test strip of claim 1, wherein the at least one passive mixing element comprises at least two passive mixing elements having substantially similar dimensions.

19. The test strip of claim 1, wherein the body comprises a wall integral with the at least one passive mixing element.

20. A method of utilizing a test strip, the method comprising:
introducing a test sample to be analyzed to a sample entry at a first end of a test strip, the test strip defining a cavity between the sample entry and a test area at a second, distal end of the test strip, wherein the test strip comprises a dissolvable bead containing at least one reagent adjacent the sample entry and at least one passive mixing element disposed within the cavity between the dissolvable bead and the test area; and
transporting at least a portion of the test sample through the cavity from the sample entry to the test area, wherein transporting at least a portion of the test sample comprises passively mixing the test sample with the at least one reagent.

21. (canceled)

22. The method of claim 20, wherein transporting at least a portion of the test sample through the cavity comprises flowing the test sample by capillary action.

23. The method of claim 20, wherein transporting at least a portion of the test sample through the cavity comprises flowing the test sample without applying an external mechanical force.

24. (canceled)

25. The method of claim 20, wherein introducing a test sample to be analyzed to a sample entry comprises introducing a sample selected from the group consisting of blood, urine, and saliva.

26. (canceled)

27. A method of forming the test strip of claim 1 comprising:
forming a body defining a sample entry, a test area, and a cavity, wherein the cavity is configured to transport a test sample from the sample entry to the test area;
providing a dissolvable bead containing at least one reagent adjacent the sample entry;
providing at least one passive mixing element within the cavity; and
configuring the at least one passive mixing element to cause passive mixing of the test sample with the reagent as the test sample is carried from the sample entry toward the test area through the cavity.

28. (canceled)

29. The method of claim 27, wherein providing a dissolvable bead containing at least one reagent adjacent the sample entry comprises at least one process selected from the group consisting of spraying, pipetting, and transferring the at least one reagent to the body.

30. (canceled)

31. The method of claim 29, further comprising lyophilizing the at least one reagent.
